(19) United States
(12) Patent Application Publication
(10) Pub. No.: US 2008/0044822 A1
(43) Pub. Date: Feb. 21, 2008

(54) NUCLEIC ACID ARRAY WITH RELEASEABLE NUCLEIC ACID PROBES

(76) Inventors: Gafur Zainiev, West Bloomfield, MI (US); Inlik Zainieva, West Bloomfield, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/465,875

(22) Filed: Aug. 21, 2006

(51) Int. Cl. C12Q 1/68

(52) U.S. Cl. 435/6

(57) ABSTRACT

A process is provided for identifying a complementary target nucleic acid. The process includes the hybridization of a nucleic acid probe to a carrier to form a nucleic acid probe-carrier complex. The complex is placed in a compartment bounded by a media permeable to the nucleic acid probe and exclusive of both the carrier and the complex. The complex is then denatured, with the nucleic acid probe transported through the media and into contact with the target nucleic acid. The nucleic acid probe hybridizes to the complementary target nucleic acid to yield a probe-target double stranded complex. A non-complementary nucleic acid probe, independent probe-target complex is returned to the compartment and given an opportunity to rehybridize to the carrier. A determination as to whether at least one of the complementary target nucleic acid or the carrier is present as a complex provides information as to probe sequences complementary to the target nucleic acid.

### FIELD OF THE INVENTION

The invention in general relates to processes and apparatuses for separating, isolating, or detecting target nucleic acids and in particular relates to processes and apparatuses suitable for regeneration of the assay.

### BACKGROUND OF THE INVENTION

Currently, nucleic acid arrays make it possible to construct in a small area solid surface such as glass, plastic or silicon an array of many thousands of DNA sequences. Nucleic acid microarray-based gene expression profiling relies on nucleic acid hybridization and the use of nucleic acid polymers, immobilized on a solid surface, as probes for complementary gene sequences. Microarrays have been used extensively to simultaneously monitor the expression of thousands of genes. Microarrays are characterized by ease of use and can be applied to large numbers of samples in parallel. Although a number of competing microarray technologies exist, two platforms (cDNA and oligonucleotide microarrays) are currently used by a majority of investigators.

With cDNA arrays, polymerase chain reaction products of cDNA clone inserts representing genes of interest are spotted systematically on nitrocellulose filters or glass slides. Spotted arrays are constructed using cDNA collections (i.e., libraries) that can be focused on genes expressed in a particular context or cell type. The primary benefit of spotted arrays is that they can be made by individual investigators, are easily customizable, and do not require a priori knowledge of cDNA sequence because clones can be used and then sequenced later if of interest. Practically speaking, however, managing large clone libraries can be a daunting task for most laboratories, and making high-quality arrays can be difficult.

Oligonucleotide microarrays use oligonucleotide probes for different genes deposited or synthesized directly on the surface of a silicon wafer in a patterned manner. Oligonucleotides offer greater specificity than cDNAs, since the oligonucleotides are tailored to minimize chances of cross-hybridization. Sequences up to 60 nucleotides are routinely used. Major advantages of this approach include uniformity of probe length and the ability to discern splice variants. The design of specific oligonucleotides has been limited by sequence availability, but the initial sequencing of the various organism genomes has made probe design easier. Oligonucleotide microarrays also provide the ability to recover samples after hybridization to a chip. This allows for a single biologic sample to be sequentially hybridized to multiple arrays. The hybridization of a test sample to an array can be detected in one of two ways. cDNA microarrays are commonly queried simultaneously with cDNAs derived from experimental and reference RNA samples that have been differentially labeled with two fluorophores to allow for the quantification of differential gene expression, and expression values are reported as ratios between two fluorescent values. Alternatively, the single color fluorescent label, where experimental mRNA is enzymatically amplified, biotin labeled for detection, hybridized to the wafer, and detected through the binding of a fluorescent compound such as streptavidin-phycoerythrin.

DNA differences between individual organisms of a particular species can provide valuable information in both a clinical and research setting. DNA resequencing is a task of sequencing a DNA region of an individual for comparison to a reference sequence associated with a specific species. DNA resequencing as a result provides information as to single nucleotide polymorphisms and mutations associated with various factors such as environmental exposure, evolutionary changes, and interspecies genetic material exchange. In a clinical setting DNA resequencing affords the possibility of tailoring medication or prophylactic treatments in response to an individual having a predisposition for a disease or condition. In a research setting, genetic changes associated with evolution, disease progression, and environmental exposure all benefit from DNA resequencing. The ability to perform genome scanning of an organism for either the whole genome or portions thereof on a routine basis would represent a significant advance in medical treatment and science. Unfortunately, the cost and complexity associated with DNA resequencing have largely precluded usage of the technique.

Thus, there exists a need for a process of target nucleic acid separation or isolation or detection that is more efficient than conventional microarrays. Additionally, there exists a need for a reusable array. With a reusable array, less sophisticated equipment is required making occasional resequencing procedures a viable process in clinical and research settings with limited resources.

### SUMMARY OF THE INVENTION

A process is provided for identifying a complementary target nucleic acid. The process includes the hybridization of a nucleic acid probe to a carrier to form a nucleic acid probe-carrier complex. The complex is placed in a compartment bounded by a first side of media permeable to the nucleic acid probe and exclusive of both the carrier and the complex. The complex is then denatured, with the nucleic acid probe transported through the media and into contact with the target nucleic acid. With the establishment of hybridization conditions, the nucleic acid probe hybridizes to the complementary target nucleic acid to yield a probe-target double stranded complex. A non-complementary nucleic acid probe, independent probe-target complex is returned to the compartment and given an opportunity to rehybridize to the carrier. A determination as to whether at least one of the complementary target nucleic acid or the carrier is present as a complex provides information as to probe sequences complementary to the target nucleic acid.

A reusable nucleic acid hybridization array channel is also provided. The channel has carrier compartment in fluid communication with a target nucleic acid compartment, and separated therefrom by a media permeable to single strand nucleic acid probes. A carrier for a nucleic acid probe is immobilized in the carrier compartment. An apparatus selectively drives the single strand nucleic acid probes between said carrier compartment and said target nucleic acid compartment.

A process is also provided for duplicating a nucleic acid array. Within a gel filled chamber bounded by an electrode, multiple copies of a nucleic acid are formed. The chamber is then brought into contact with a second gel filled chamber bounded by a second electrode. An electrical potential is formed across the electrodes to induce electrophoretic migration of a portion of the nucleic acid from said first chamber to said second chamber. The separation of the chambers yields the duplicate array when the process occurs in parallel for multiple isolated chambers.

### BRIEF DESCRIPTION OF THE DRAWINGS

The present invention is further detailed with respect to the following figures that illustrate particular embodiments of the present invention. These figures are not intended to limit the invention to that specifically disclosed therein but rather to provide illustration as to the substance of the appended claims.

FIGS. 1(a)-(p) schematically depict a sequence of procedural steps to operate an inventive array with releasable nucleic acid probes;

FIG. 2 depicts a multi-channel DNA array according to the present invention;

FIGS. 3(a)-(d) depict various embodiments of multiple channel electrophoretic array chambers according to the present invention in cross-section schematic view;

FIG. 4 is a cross-sectional schematic that depicts a series of chambers with conductive wires providing electrical leads to each of an electrode pairs;

FIG. 5 is an explodes view of a combination of plates to form an array of electrophoretic chambers as shown in FIG. 4;

FIGS. 6(a)-(d) schematically depict a sequence of procedural steps to form a duplicative array with nucleic acid migration from a template array to create a copy array; and

FIGS. 7(a)-(r) schematically depict a sequence of procedural steps to operate an electrophoretic diagnostic according to the present invention with a carrier compartment having a carrier immobilized within a gel and a target nucleic acid compartment in which a target nucleic acid is also immobilized.

### DETAILED DESCRIPTION OF THE PREFERRED EMBODIMENTS

The present invention has utility in the separation, isolation or detection or combination of these outcomes for a target molecule that is a nucleic acid multimer or bound to a nucleic acid multimer in a way that does not preclude a complementary nucleic acid probe from binding the multimer. Both prior art DNA microarrays carry single stranded DNA probes immobilized as spots on the surface of a microarray with hybridization of single strand DNA targets to the immobilized single strand DNA probes as a means for detection. The present invention introduces a third type of nucleic acid molecule, namely a single strand nucleic acid carrier that is complementary to a single strand nucleic acid probe such that under appropriate conditions a completely complementary double strand nucleic acid structure is formed between the carrier and the probe. The present invention in utilizing a carrier oligonucleotide for a nucleic acid probe thereby allows such probes to be untethered molecules in solution. At the same time, nucleic acid target molecules are utilized as free molecules in solution, per the prior art, or alternatively immobilized on a solid surface or embedded in a porous media. The carrier molecule of the present invention provides considerable flexibility in terms of usage and illustratively is immobilized on a solid surface, provided as a free molecule in solution or embedded in porous media capable of fluid communication with a complementary nucleic acid probe. As a result, the present invention offers a degree of flexibility in operation, simplified manufacture and operation, and in regard to certain embodiments allows one to regenerate the inventive array for subsequent usage.

According to the present invention it is possible to prepare a complex of carrier and nucleic acid probe by first preparing a long double stranded nucleic acid which after treatment with specific restriction enzymes the second stand becomes a number of short nucleic acid strands hybridized to an elongated carrier strand. This procedure facilitates manufacture of numerous copies of nucleic acid probes by first amplifying long and repetitive double strand nucleic acid molecules and then treating such long double strand nucleic acid molecules with the appropriate restriction enzymes.

The present invention relies on a carrier capable of uniquely and reversibly binding a nucleic acid probe. In an inventive array, carriers are preferably isolated dimensionally in space or on a substrate. It is appreciated that in an array according to the present invention with carriers immobilized on a surface or within a porous matrix, nucleic acid probes can be harvested from a random mixture of short oligonucleotides, having a length of between 5 and 50 bases. Oligonucleotides harvested from the random mixture can be used as nucleic acid probes for subsequent hybridization and use in assays.

As used herein, a “carrier” is defined as a substance able to uniquely and reversibly bind to a nucleic acid probe and includes complementary nucleic acid sequences, pore structures, and other organic molecules. It is appreciated that a carrier need not be a nucleic acid and instead can be formed by a complex of non-nucleic acid molecules generating a gel-like structure such that a nucleic acid probe is immobilized on the surface or internal to the gel-like body. An example of this is found in Proudnikov et al., Anal. Biochem. 1998, 259, 34. Alternatively, a carrier is a nucleic acid molecule to which is attached a non-nucleic acid moiety. As used herein, such a carrier is considered a mixed carrier and is readily provided in solution, immobilized to a surface or within porous media. Non-nucleic acid molecules suitable for bonding to a nucleic acid carrier according to the present invention are virtually unlimited and can include within the non-nucleic acid moiety a function such as a binding site to a substrate, a recognition site for a probe, a spectroscopically active label, or combinations thereof.

The arrangement of carriers in space so as to provide an inventive array includes a number of options in manufacture and operation. By way of example, carriers are coupled together to form an elongated strand. Preferably, the identity and position of each carrier along the strand is known. More preferably, spacer segments are provided intermediate between carriers along a strand so as to disfavor steric hindrance with probes pairing with the carrier sequences along the strand. It is appreciated that the specific inclusion of restriction sites within linker segments of the strand or knowledge as to such sites within carrier nucleic acid sequences provides for subsequent modification to replace a given carrier with a new carrier having different specificity. The ability to produce an elongated strand of carriers secured to a substrate by one or more strand termini creates an interaction environment with a probe in solution that is largely free of substrate surface interaction and the hindrances to probe-carrier complexation associated with a monolayer of probes immobilized on a substrate spot as in a conventional DNA microarray. As a result, an elongated strand of carriers provides particular advantages in the use of nucleic acid probes having a length exceeding 40 nucleic acid bases and is functional beyond 60 nucleotide bases and is generally considered an upper limit in a conventional micro array.

Carriers are immobilized in two dimensions on a surface and are similar to spotting associated with a conventional microarray such that the position of each carrier can be identified. According to the present invention, an extension of conventional two-dimensional surface spotting is the arrangement of carriers in a there-dimensional space such as embedded in porous media to provide a higher carrier density while retaining the ability to identify the position of carriers. Preferably, porous media in which carriers are embedded in three dimensions is optically transparent to facilitate position identification for a given carrier through spectroscopic interrogation from various orientations.

In those instances when a carrier is not a complex of non-nucleic acid molecules forming a gel-like structure, the carrier is readily arranged temporally. Temporal arrangement of carriers occurs with the carriers in succession passing through a detector that identifies a carrier based on a parameter illustratively including specific characteristics of the carrier as to size, conformation, an attached label, or combinations thereof; a time schedule; or a predetermined order of carriers passing through the detector.

The ability to bind nucleic acid target species immobilized on a solid surface and/or trapped in a porous media such as an electrophoretic gel according to the present invention offers advantages requiring less steps of purification. Likewise, nucleic acids targets immobilized on the surface of a nucleic acid microarray are readily identified with nucleic acid probes according to the present invention. Still a further variant to facilitate operation of the present invention involves immobilizing target nucleic acid molecules on particles that greatly facilitate subsequent separation. Such particles illustratively include metals, paramagnetics, semiconductors, and polymers.

In one mode of operation, a single strand target nucleic acid is hybridized with single strand nucleic acid probes in solution and thereafter a double stranded complex of target nucleic acid-nucleic acid probe is separated from unassociated single strand nucleic acid probes. These free single strand nucleic acid probes are then hybridized to carriers remote from the target nucleic acid-probe nucleic acid double stranded complexes. In this mode of operation, it is appreciated that target nucleic acids can be immobilized on a solid surface, illustratively including the aforementioned paramagnetic particles, another nucleic acid microarray bonded to another molecular species; or embedded in a gel such as through a process commonly used for electrophoretic separation. In visualization results in this mode of operation, a user either visualizes carriers that remain unassociated with the complementary probe; or in the alternative visualizes carriers that are complexed to the complementary probe nucleic acid thereby indicating that the nucleic acid target for the probe nucleic acid now complexed to its carrier had no target nucleic acid sequence with which to bind.

An alternate mode of operation for the inventive assay involves immobilizing to a surface or within a porous media carrier with probe nucleic acids complexed thereto. The probe nucleic acids are released in the appropriate volume of solution and are then free to search for a complementary target nucleic acid. The decomplexation stimulus for the carrier-probe nucleic acid pair illustratively includes thermal energy, an electric field and pH change.

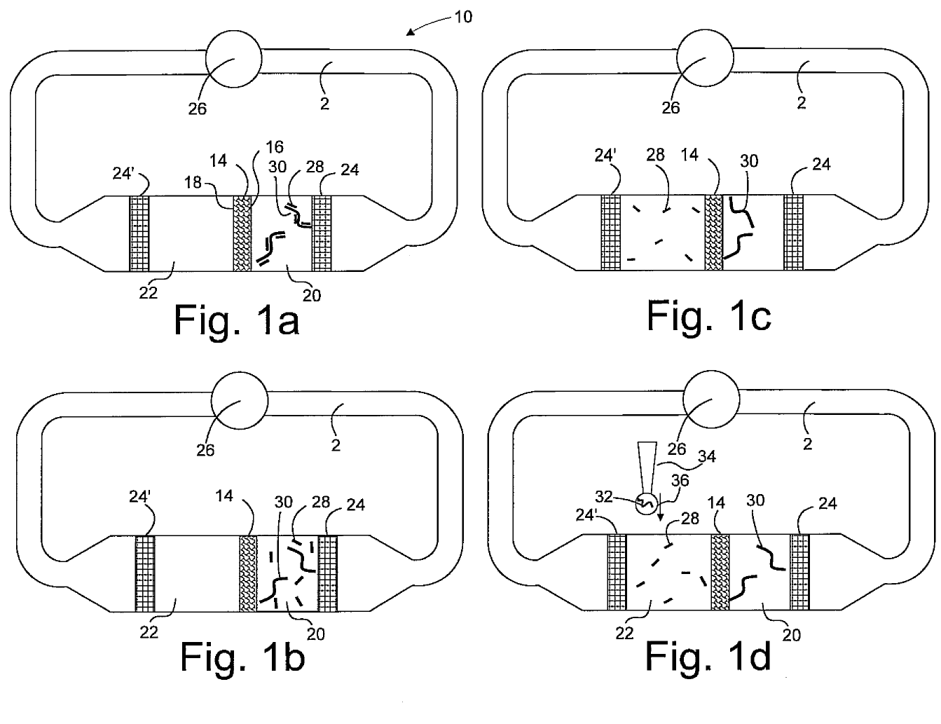

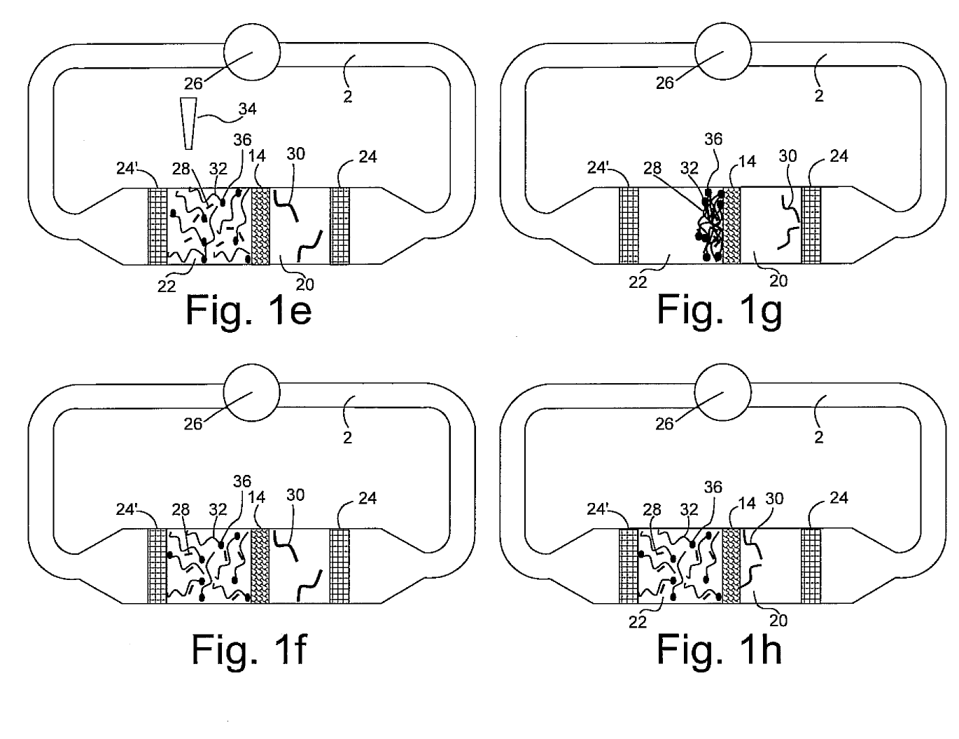

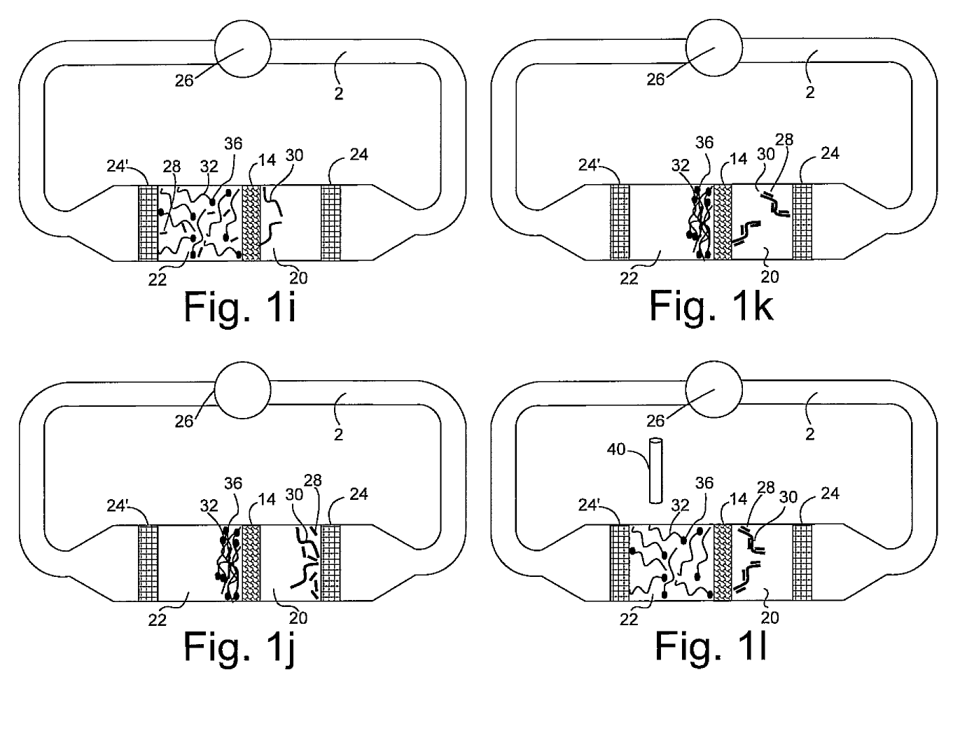

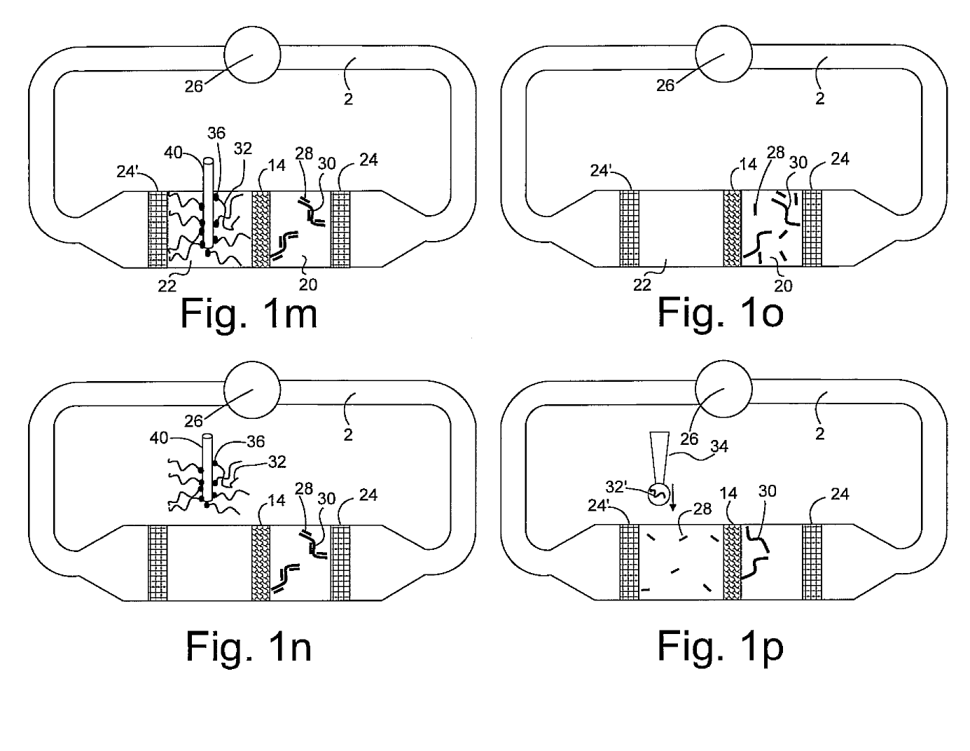

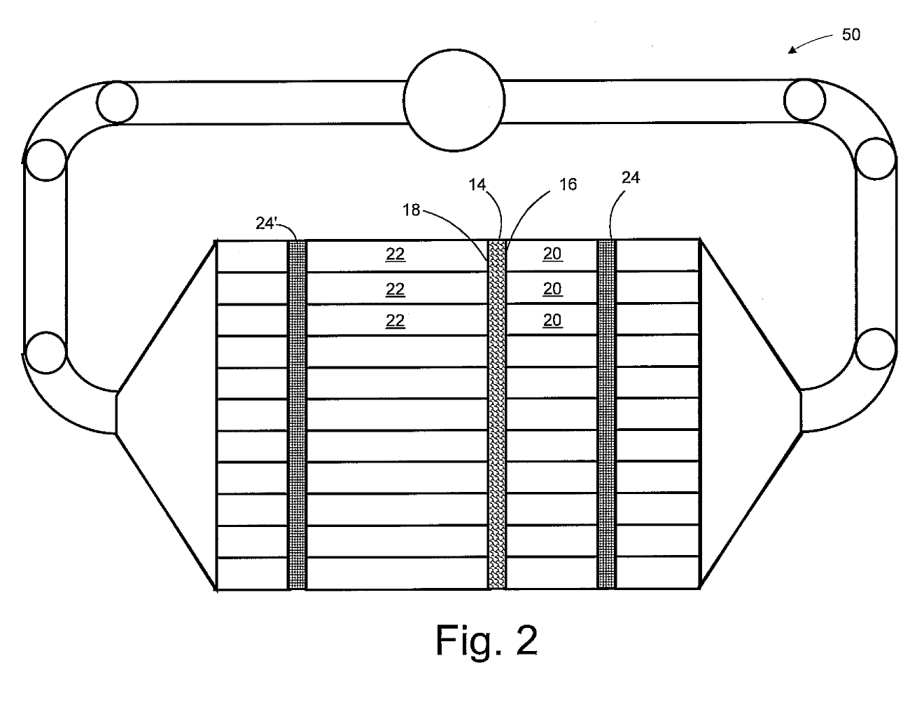

Referring now to FIG. 1, a series of steps are depicted in the operation of a single channel of an inventive array. While the process steps depicted in FIG. 1 correspond to an array in which solution is moved between array components, it is appreciated that an electric field likewise moves charged probe nucleic acids between compartments that remain filled with electrolyte solution. Electrolytic operation is further detailed with respect to FIG. 2. In instances where an electric field is used to move probe nucleic acids, the observable measurement need not be labeled a probe nucleic acid but rather a measurement of ionic current or measurement of probe nucleic acids through the media independent of specifics of nucleic acid probe movement.

The channel depicted in FIG. 1 is a single channel device depicted generally at 10 of an inventive array. The device 10 has a channel 12 in fluid communication with porous media 14 that is permeable to nucleic acid probes 28 while exclusive of carriers 30 and target nucleic acids 32. The porous media 14 has a first side 16 and a second side 18. The porous media 14 defines a boundary between a carrier compartment 20 and a target nucleic acid compartment 22. Optionally, the carrier compartment 20 is bounded by a fluid communicative porous media 24, the media 24 being exclusive of carrier 30, probe nucleic acid 28, and target nucleic acid 32 species. Likewise, optionally a fluid communicative porous media exclusive of probe nucleic acid, carrier and target nucleic acid species is provided at 24′ to bound the target nucleic acid compartment 22. A nucleic acid movement-inducing apparatus 26 is provided to urge probe nucleic acid species 28 between compartments 20 and 22, and vice versa. The apparatus 26 has an identity dictated by the type of force used to urge probe nucleic acids between compartments. By way of example, in instances where fluid flow induces probe nucleic acid 28 to move between compartments 20 and 22, the apparatus 26 is a pump. Alternatively, in instances where nucleic acid probe 28 moves under the influence of an electrostatic potential, the apparatus 26 is a power supply with electrodes inducing a potential between porous media 24 and 24′. For illustrative purposes apparatus 26 is detailed throughout FIG. 1 as a pump moving probe nucleic acids 28 by way of fluid flow. In FIG. 1(a), the nucleic acid probe 28 is present in multiple copies but hybridized to a carrier 30 retained within carrier compartment 20. The carrier 30 is appreciated to be present within compartment 20 as a solution species, adhered to porous media 24 from side 16 of porous media 14, or embedded within porous media 24 or 14.

As shown in FIG. 1(b), a complex of nucleic acid probe 28-carrier 30 is denatured to liberate nucleic acid probe 28 into the solution volume of carrier compartment 20. Denaturing of a nucleic acid probe 28-carrier 30 complex occurs through a variety of techniques illustratively including heating, pH change, and induction of a voltage potential.

As shown in FIG. 1(c), nucleic acid probes 28 are induced to migrate into side 16 of the porous media 14 to arrive in target nucleic acid compartment 22. As depicted in this figure, the flooding of compartment 22 through a pumping action on channel 12 or the drawing of a vacuum in compartment 22 carries nucleic acid probes 28 into target nucleic acid compartment 22.

As shown in FIG. 1(d), target nucleic acid molecules 32 are delivered into target nucleic acid compartment 22 by way of a dispenser 34, the dispenser illustratively including a micropipette, a microdispenser, or the like. It is appreciated that the single stranded target nucleic acid molecules 32 are readily introduced into compartment 22 prior to movement of nucleic acid probes 28 being introduced into compartment 22. In the embodiment depicted in FIG. 1, the single stranded target nucleic acid molecules 32 are each immobilized to a paramagnetic particle 36.

Referring now to FIG. 1(e), the target nucleic acid molecules 32 adhere to paramagnetic particle 36 and are allowed to interact with multiple copies of nucleic acid probe 28. As shown in FIG. 1(f), it includes conditions under which a nucleic acid probe 28 hybridizes to a complementary single strand target nucleic acid 32 to form a double strand complex. Hybridization techniques and conditions are well known to the art and illustratively include thermal cooling, chances in ionic strength, and pH change. At this point, the contents of the target nucleic acid compartment 22 can be removed and assayed for complexation between a given target nucleic acid and a nucleic acid probe 28. Such analysis is facilitated by a fluorescently labeled nucleic acid probe. The multiple potential target nucleic acids, some of which are complexed to nucleic acid probes while others may not be targeted to nucleic acid probes, are readily resolved through conventional techniques such as chromatography or electrophoresis. However, in a preferred embodiment as depicted in FIG. 1(g), the solution is transferred from compartment 22 back to carrier compartment 20 with the net result that should the nucleic acid probe 28 be complementary to a portion of target nucleic acid 30, then the nucleic acid probes 28 remain in compartment 22. Direct measurement of carrier 30 under hybridization conditions by conventional techniques such as fluorescent dyes allows one to determine if nucleic acid probes 28 have returned to carrier compartment 20 and hybridized to carrier 30 thereby indicating that the nucleic acid probe 28 is not complementary to target nucleic acid 32. In the event that nucleic acid probe 28 is not complementary to target nucleic acid 32, flushing the target nucleic acid 32 from compartment 22 along with any optional paramagnetic particles 36 returns the channel 10 to a start position of FIG. 1(a) with the optional removal of residual fluorescent dye therefrom.

In the event that the nucleic acid probe 28 is complementary to the target nucleic acid 30, preferably the target nucleic acid compartment 22 is again filled with solution as depicted in FIG. 1(h). Denaturing any complexes of nucleic acid probe 28 with target nucleic acid 30 occurs under conditions similar to those created in FIG. 1(b), as depicted in FIG. 1(i).

The subsequent steps involve the separation of the target nucleic acids 32 introduced in FIG. 1(d) from the nucleic acid probes 28 that previously hybridized thereto. While it is appreciated that numerous techniques such as chromatography, electrophoresis, and taking advantage of the attached paramagnetic particles 36 are operative to create such a separation external to channel 10 in a preferred embodiment depicted in FIG. 1(j), the now free nucleic acid probes 28 are transported from the target nucleic acid compartment 22 to the carrier compartment 20. By creating hybridization conditions in carrier compartment 20, nucleic acid probe 28 and carrier 30 again form a stable complex. Complexation conditions between nucleic acid probe 28 and carrier 30 are those associated with forming the structures depicted in FIG. 1(a) or the target nucleic acid-nucleic acid probe double stranded structures of FIG. 1(f). With the formation of nucleic acid probe 28-carrier complex as depicted in FIG. 1(k), the target nucleic acid compartment 22 is then refilled to return the single stranded target nucleic acids 32 with attached paramagnetic particles 36 to solution within the compartment 22 as shown in FIG. 1(l). The introduction of a magnet 40 into the compartment 22 causes paramagnetic particles 36 to adhere to the magnet 40, as depicted in FIG. 1(m). Withdrawal of the magnet 40 from the compartment 22 causes the magnet 40 to carry therewith the paramagnetic particles 36 in the attached target nucleic acids 32, as depicted in FIG. 1(n).

The withdrawal of solution from target nucleic acid compartment 22 returns the channel 10 to an original state depicted in FIG. 1(a), as shown in FIG. 1(o).

Subsequent decomplexation of nucleic acid probes 28 and carrier 30 and the filling of target nucleic acid component 22 leaves the channel 10 ready to receive a new target nucleic acid sample 32′. The new nucleic acid sample 32′ optionally includes target nucleic acids attached to paramagnetic particles 36, as shown in FIG. 1(p) which mirrors the condition as depicted in FIG. 1(d).

Referring now FIG. 2, a schematic of a multiple channel array according to the present invention is depicted where like numerals correspond to those previously described with respect to FIG. 1. A two-dimensional or three-dimensional array of channels is provided with multiple carrier compartments 20 and target nucleic acid components 22. An advantage of a multiple channel array 50 as depicted in FIG. 2 is that multiple compartments 20 and 22 facilitate high throughput automation. Preferably, target nucleic acids provided on an immobilized DNA chip yield a high throughput genotyping system. Additionally, since nucleic acid probes are shuttled between compartments 20 and 22 and not expended, an inventive multiple channel array is reusable further facilitating automation and/or usage in poorly equipped research or medical laboratories.

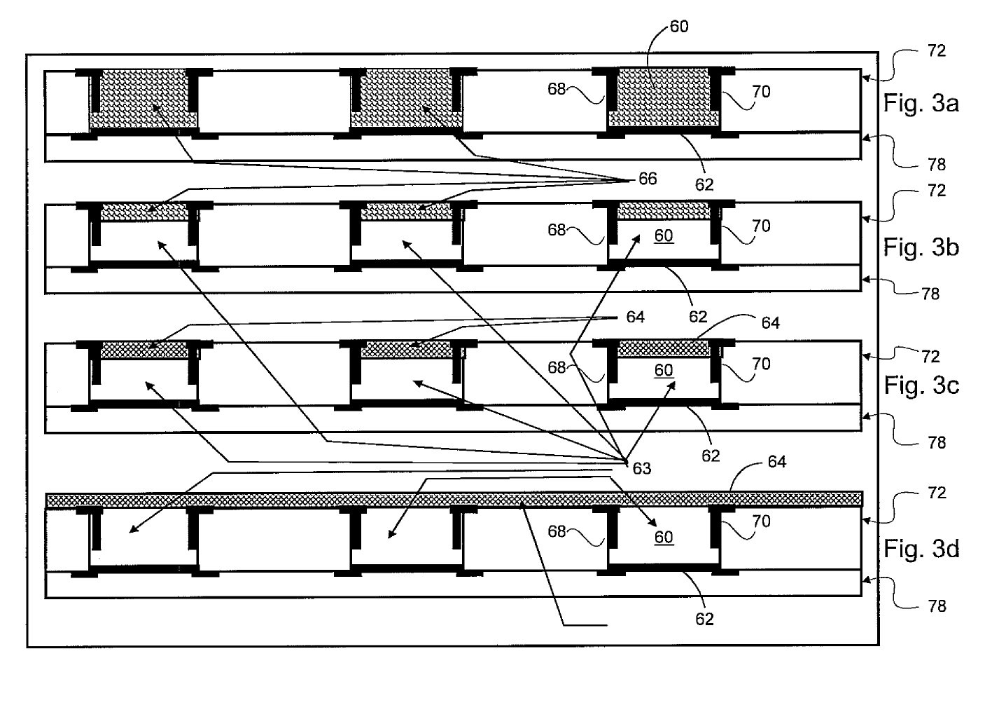

An electrophoretic multiple channel inventive array has a carrier chamber 60 bounded by a terminal electrode 62. Chamber 60 precludes a carrier or target nucleic acid from leaving the chamber 60 through resort to a porous material 64 through which a full length carrier or target nucleic acid cannot pass or alternatively through embedding the carrier or target nucleic acid within a gel 66. It is appreciated that a porous material bounds an aqueous solution 63 within chamber 60 while a gel 66 bounds either an aqueous solution containing full length carrier or target nucleic acids or alternatively entirely fills the chamber 60. The chamber 60 also includes a pair of laterally spaced electrodes 68 and 70. Various embodiments of a multiple channel electrophoretic array chamber are depicted in FIGS. 3(a)-3(d).

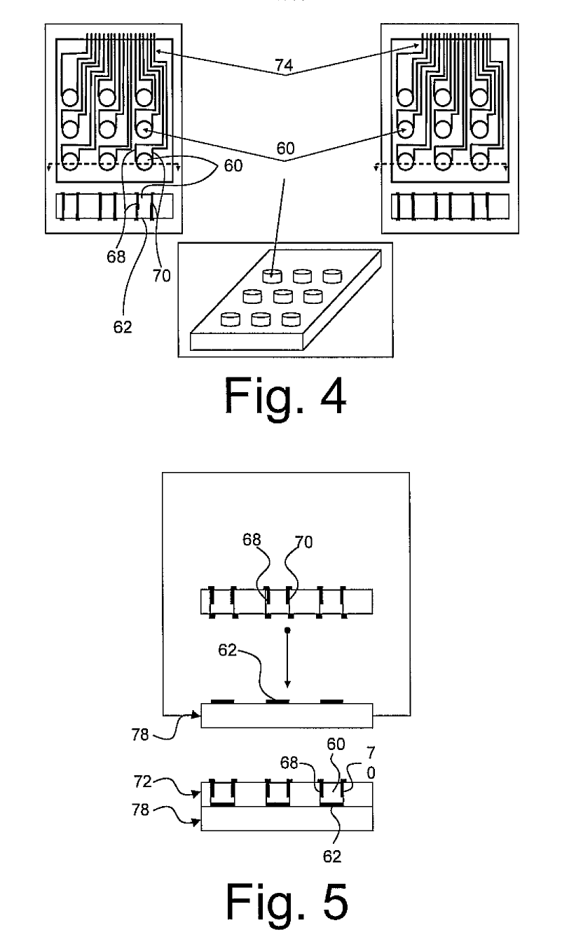

As shown in FIG. 4, a first plate such as that represented at 72 in FIG. 3(d) forms a series of chambers with conductive wires 74 providing electrical leads to each of the electrode pairs 68 and 70 where like numbers correspond to those used with respect to FIG. 3.

FIG. 5 depicts in exploded view a combination of plates 72 and plate 78 to form an array of electrophoretic chambers with the inclusion of a porous material 64 or gel 66 to bound the exposed opening to chamber 60.

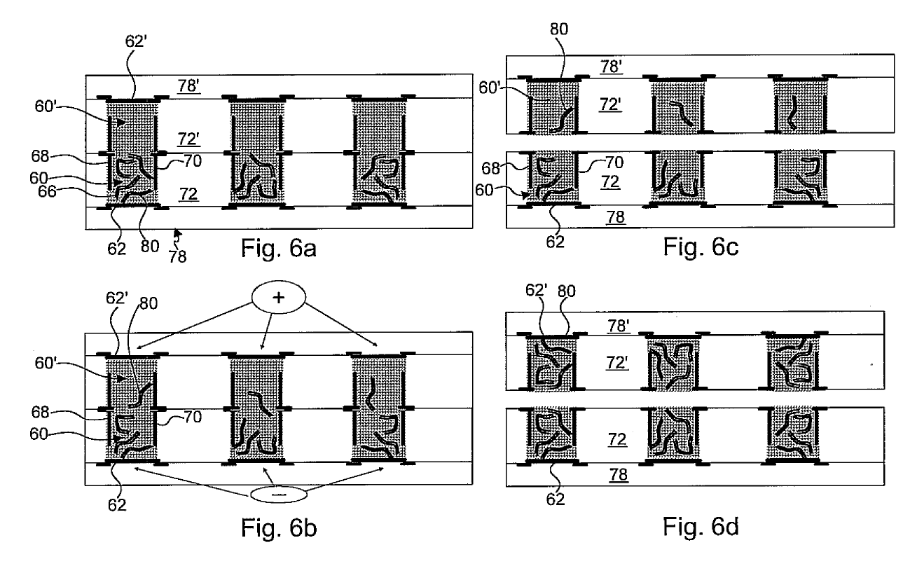

The use of an inventive electrophoretic array to form a duplicate inventive array is depicted schematically in FIGS. 6(a)-(d) in an instance where an inventive chamber contains a target nucleic acid molecule or carrier of interest 80 where like numerals correspond to those used with respect to FIGS. 3-5. Nucleic acid 80 is amplified by conventional techniques such as PCR within the chamber 60 to form multiple copies. While the gel retains sample 80 therein, PCR nucleic acid residue reagents readily diffuse within the gel. With multiple copies 80 of a sample nucleic acid present within chamber 60 embedded in gel 66, mirror image plates 72′ and 78′ having terminal electrodes 62′ are brought into contact with plate 72. As depicted in FIG. 6(b), a voltage is applied across electrodes 62 and 62′ to transfer the nucleic acid 80 from chamber 60 to chamber 60′. After transfer of sample nucleic acid 80 to chamber 60′, plate 72 is separated from plate 70 and electrophoretic necessarily terminated therebetween, as depicted in FIG. 6(c). The modest quantity of nucleic acid sample 80 within plate 60′ is multiplied by polymerase chain reaction (PCR) within chamber 60′ so as to yield a duplicate plate combination 72 and 78 corresponding to nucleic acid sample 80 within chamber 60. In this way a complete copy of a set of sample nucleic acids is so produced.

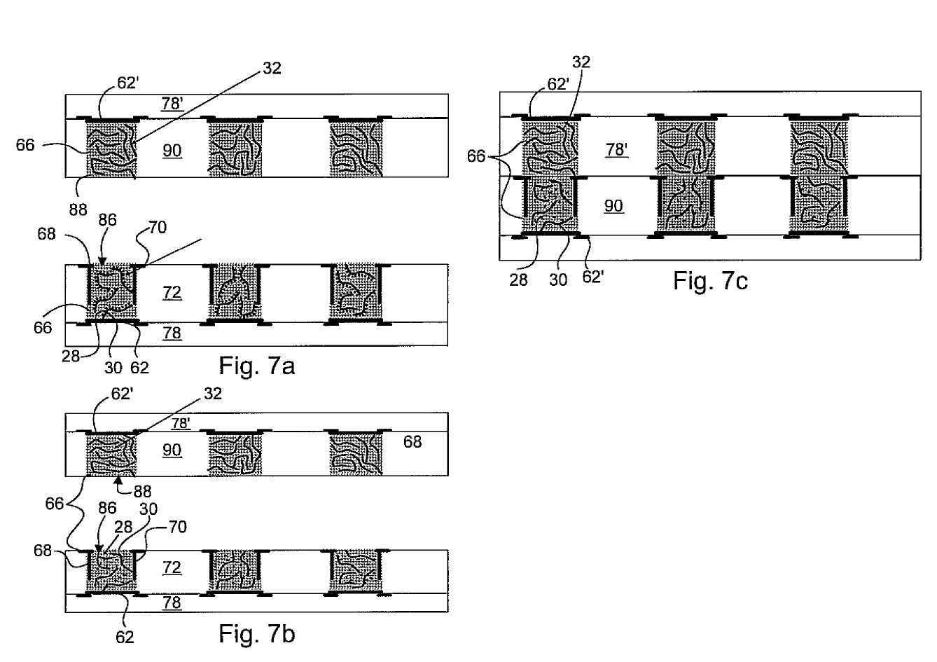

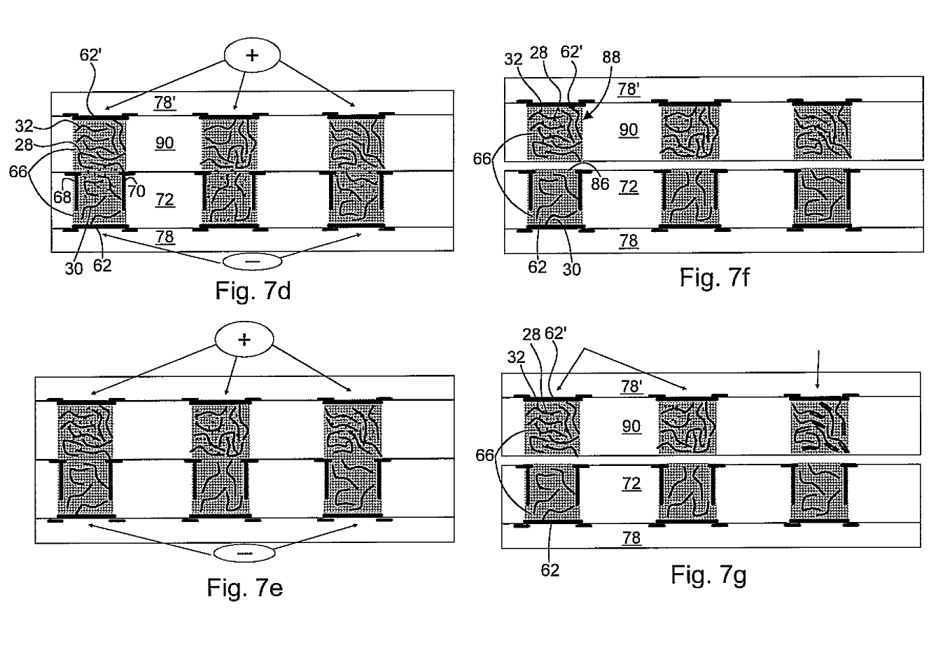

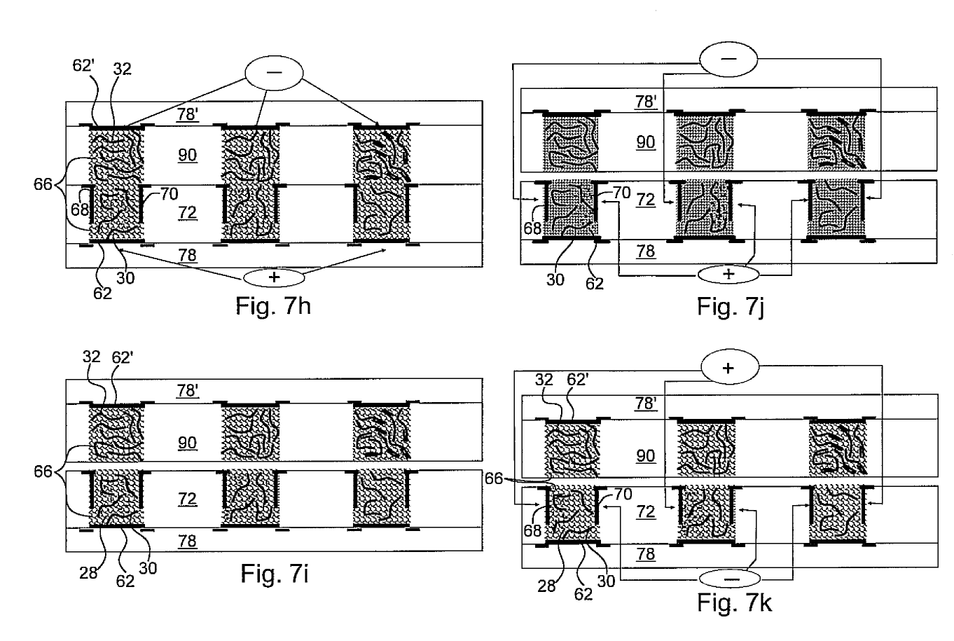

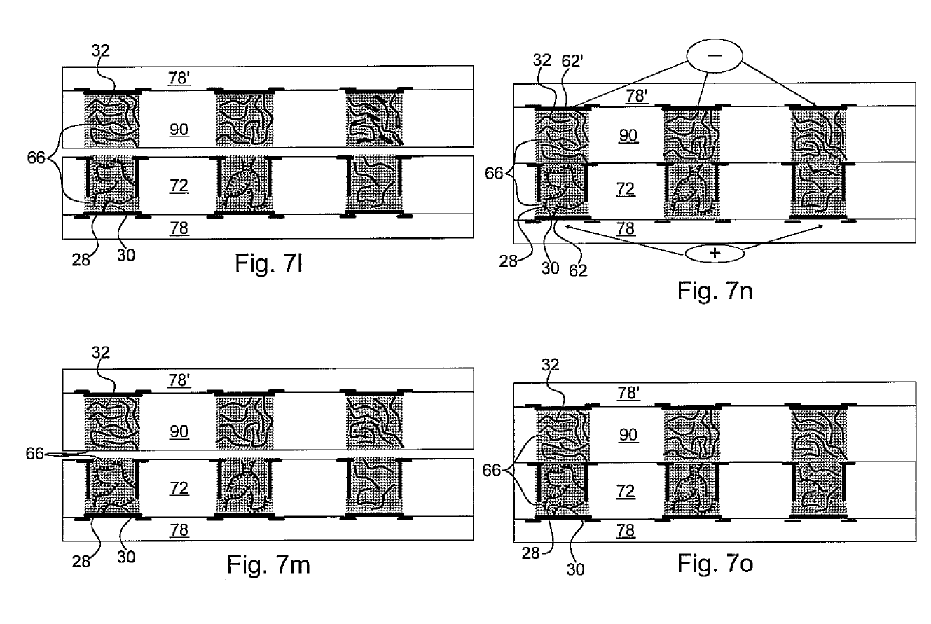

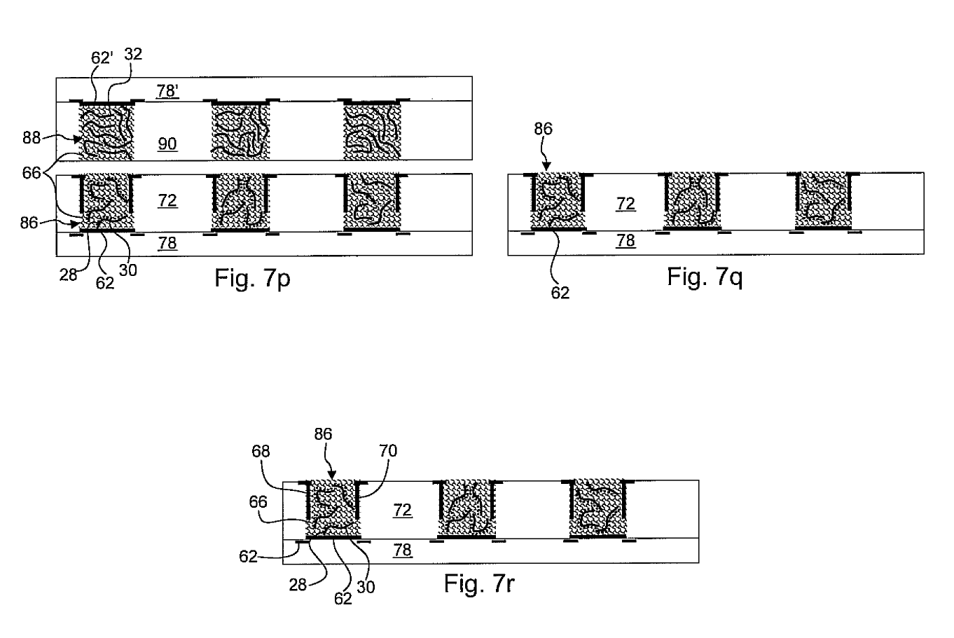

Referring now to FIGS. 7(a)-(r), the operation of an electrophoretic diagnostic according to the present invention is detailed in the instance where a carrier compartment includes a carrier immobilized within a gel and a target nucleic acid is also immobilized within a gel of a target nucleic acid compartment. A diagnostic array includes plates 72 and 78 defining a terminal electrode 62 and side electrodes 68 and 70 as detailed with respect to the previous figures to define a chamber 60 containing a number of short nucleic acid probes 28 hybridized to long single stranded carrier 30. Gel 66 fills the chamber 60 to cumulatively define a carrier compartment 86. Target nucleic acid 30 is placed in gel 66 to form a target nucleic acid compartment 88. The compartment 88 is bound by a terminal electrode 62′ formed by combining plate 78′ with perforated plate 90. As depicted in FIG. 7(b), the complex of nucleic acid probes 68 with single stranded carrier 30 is exposed to conditions to cause the complex to denature from double stranded to single stranded carrier 30 and nucleic acid probes 28. Plate 90 and plate 78′ affixed thereto are then brought into contact with plate 72. With the application of electrostatic potential between electrodes 62 and 62′, the nucleic acid probes 28 are induced to migrate from the carrier compartment 86 into the target nucleic acid compartment 88. As shown in FIG. 7(e), at the end of this electrophoretic process carrier compartment 86 no longer contains nucleic acid probes 28 and all such probes 28 are now within the target nucleic acid chamber 88. As shown in FIG. 7(f), plate 90 is then separated from plate 72. The separated set of target nucleic acid chambers 88 are now exposed to conditions suitable to induce hybridization between target nucleic acids 34 and nucleic acid probes 28. Complementary target nucleic acid 34 and nucleic acid probes 28 hybridize to form a double stranded complex as shown in FIG. 7(g). Thereafter, plate 90 is returned to contact with plate 72 and the electrophoretic polarity reversed relative to that used in FIG. 7(d) so as to drive any unhybridized nucleic acid probes 28 from target nucleic acid compartment 88 into carrier compartment 86. As shown in FIG. 7(i), plate 90 is again separated from plate 72 and horizontal electrophoresis performed on nucleic acid probes 28 within the carrier chambers 86. Reverse polarity electrophoresis to move nucleic acid probes 28 within carrier compartments 86 is performed as depicted in FIG. 7(k). An ionic conductivity measurement is provided for each carrier compartment 86 based on the amount of nucleic acid probes 28 traveling therein under the influence of forward and reverse electric fields of FIGS. 7(j) and (k). Carrier compartments 86 are then subjected to conditions under which hybridization can occur between carrier 30 and nucleic acid probes 28 as depicted in FIG. 7(l).

The target nucleic acid compartments 88 are then exposed to conditions sufficient to denature double stranded complexes that exist between target DNA 34 and nucleic acid probes 28. Plates 90 and 72 are again brought into contact and the potential established between electrodes 62 and 62′ with the same polarity as that provided in FIG. 7(h) to induce migration of nucleic acid pairs 28 complementary to target nucleic acid 34 into carrier compartment 86. With all nucleic acid probes 28 returned to carrier compartments 86, the electrophoretic potential between electrodes 62 and 62′ is discontinued as shown in FIG. 7(o). Plates 90 and 72 are then separated with a target nucleic acid 34 content of compartments 88 being that of the original as depicted in FIG. 7(a). In FIG. 7(q), the carrier compartments 86 are subjected to hybridization conditions to create complexes between the nucleic acid probes 28 that were complementary to target nucleic acid 34 thereby returning the contents of carrier compartment 86 to an original state that is ready for coupling with a new set of target nucleic acids while the target compartments 88 are likewise suitable to contact with a new set of carrier compartments containing different nucleic acid probes.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A process of identifying a probe sequence to complementary target nucleic acid comprising:
hybridizing a nucleic acid probe complementary to the complementary target nucleic acid to a carrier yield a probe-carrier complex;
placing said probe-carrier complex into a compartment bounded by a first side of media permeable to said nucleic acid probe and exclusive of said carrier and said complex;
denaturing said complex;
allowing said nucleic acid probe to migrate through said media to a second side;
bringing the complementary target nucleic acid into contact with said nucleic acid probe;
providing hybridization conditions such that said nucleic acid probe has an opportunity to hybridize to the complementary target nucleic acid to yield a probe-target complex;
allowing said nucleic acid probe the opportunity to rehybridize to said carrier when said nucleic acid probe is not part of said probe-target complex; and
determining whether at least one of the complementary target nucleic acid or said carrier is present as said probe-carrier complex or said probe-target complex to identify a probe sequence to complementary target nucleic acid.

2. The process of claim 1 further comprising denaturing said probe-target complex and returning all of said nucleic acid probe to the first side of said media to reform said probe-carrier complex.

3. The process of claim 1 wherein said nucleic acid probe migrates to the second side of said media via solution flow from said carrier compartment through said media.

4. The process of claim 1 wherein said nucleic acid probe migrates to the second side of said media by establishing an electrophoretic potential across said media.

5. The process of claim 1 wherein said carrier is monitored through the presence of said probe-carrier complex with fluorescence subsequent to providing hybridization conditions for said nucleic acid probe and the complementary target nucleic acid to yield the probe-target complex.

6. The process of claim 1 wherein the complementary target nucleic acid is attached to a paramagnetic particle.

7. The process of claim 1 wherein the complementary target nucleic acid is bound within a gel.

8. The process of claim 1 wherein said carrier is a strand hybridized to multiple copies of said nucleic acid probe.

9. The process of claim 1 wherein said nucleic acid probe is attached to a detectable label.

10. The process of claim 1 further comprising repetition with the steps of claim 1 simultaneously in a plurality of isolated carrier compartments each containing a single nucleic acid probe that varies in identity from said nucleic acid probe.

11. A reusable nucleic acid hybridization array channel comprising a carrier for a nucleic acid probe immobilized in a carrier compartment in fluid communication with a target nucleic acid compartment;
a media permeable to single strand nucleic acid probes intermediate between said carrier compartment and said target nucleic acid compartment; and
an apparatus for selectively driving said single strand nucleic acid probes between said carrier compartment and said target nucleic acid compartment.

12. The channel of claim 11 wherein at least one of said carrier compartment and said target nucleic acid compartment comprises a gel.

13. The channel of claim 11 wherein said carrier is immobilized within said carrier compartment in a form selected from the group consisting of: adhered to a surface and incorporated into a gel.

14. The channel of claim 11 wherein said carrier is a linear strand hybridizing to multiple copies of said nucleic acid probe.

15. The channel of claim 11 wherein said carrier compartment and said target nucleic acid compartment are bounded by a pair of electrodes and said source is a power supply.

16. The channel of claim 15 further comprising a second electrode pair forming a potential gradient only within said carrier compartment.

17. The channel of claim 11 wherein said apparatus is a pump transferring solution between said carrier compartment and said target nucleic acid compartment,

18. A reusable nucleic acid array comprising: a plurality of channels according to claim 11 extending in two dimensions.

19. The array of claim 1I wherein said plurality of channels extend in three dimensions.

20. A process for duplicating a nucleic acid array comprising:
forming multiple copies of the nucleic acid within a gel filled chamber bounded by an electrode;
placing said chamber in contact with a second chamber filled with gel and bounded by a second electrode;
inducing electrophoretic migration of a portion of the nucleic acid from said first chamber to said second chamber; and
separating said second chamber from said first chamber.

21. The process of claim 20 further comprising amplifying the portion of nucleic acid within said second chamber to form multiple copies of the portion of the nucleic acid.

22. The process of claim 21 wherein the amplification is by polymerase chain reaction.
